(19) United States
(12) Patent Application Publication
(10) Pub. No.: US 2005/0148867 A1
(43) Pub. Date: Jul. 7, 2005

(54) Injector with changeable syringe constants

(75) Inventor: Charles S. Neer, Cincinnati, OH (US)

Correspondence Address: WOOD, HERRON & EVANS, L.L.P., 2700 Carew Tower, 441 Vine St., Cincinnati, OH, 45202, US

(73) Assignee: Liebel-Flarsheim Company, Cincinnati, OH (US)

(21) Appl. No.: 10/749,894

(22) Filed: Dec. 31, 2003

(51) Int. Cl. A61B 6/00

(52) U.S. Cl. 600/431

(57) ABSTRACT

An intravenous contrast injector which stores different syringe definitions in firmware includes a software routine that permits a service technician to update the syringe definitions without requiring the installation of new firmware. These syringe definitions can then be used when the injector performs operational routines such as filling a syringe.

### FIELD OF THE INVENTION

The present invention relates to injectors for injecting fluid into patients.

### BACKGROUND OF THE INVENTION

In many medical environments, a medical fluid is injected into a patient during diagnosis or treatment. One example is the injection of contrast media into a patient to improve Optical Imaging, Nuclear Medicine, CT, Angiographic, Magnetic Resonance or Ultrasound imaging, or any diagnostic imaging or therapeutic application using a powered, automatic injector.

Injectors suitable for these and similar applications typically must use a relatively large volume syringe and be capable of producing relatively large flow rates and injection pressures. For this reason, injectors for such applications are typically motorized, and include a large, high mass injector motor and drive train. For ease of use, the motor and drive train are typically housed in an injection head, which is supported by a floor, wall, or ceiling mounted arm.

The injection head is typically mounted on the arm in a pivotal manner, so that the head may be tilted upward (with the syringe tip above the remainder of the syringe) to facilitate filling the syringe with fluid, and downward (with the syringe tip below the remainder of the syringe) for injection. Tilting the head in this manner facilitates removal of air from the syringe during filling, and reduces the likelihood that air will be injected into the subject during the injection process. Nevertheless, the potential for accidentally injecting air into a patient remains a serious safety concern.

In addition to the injection head discussed above, many injectors include a separate console for controlling the injector. The console typically includes programmable circuitry which can be used for automatic, programmed control of the injector, so that the operation of the injector can be made predictable and potentially synchronized with operations of other equipment such as scanners or imaging equipment.

Within this environment, the physical parameters of a particular syringe, known as syringe constants, have previously been presumed to remain uniform and constant for a particular model of syringe. These physical parameters includes such features as syringe inner diameter, syringe stroke length, and syringe volume. However, variations can occur based on factors such as manufacturing variances, resin changes, syringe design changes, and changes in system requirements. For example, if a more rigid plastic is selected for a syringe, then its walls can be thinner than if a softer plastic were used. Accordingly, if the outside dimensions of that syringe remained the same, the inner diameter and volumetric capacity of the syringe would differ from that of a syringe made with the softer plastic.

In the past, accommodating syringe variations was often difficult, time-consuming and expensive. The firmware for controlling the injector typically includes definitions of permitted syringes. Thus, any changes to the physical parameters of a syringe required entirely new firmware such as EPROMS, orother non-volatile storage, to be created which then required service personnel to visit each site having an injector in order to replace the outdated EPROM.

Some injector systems require the operator to provide information about a syringe's physical characteristics as part of performing an operational routine with the injector system. This approach, however, does not address the need for a service technician to be able to easily update syringe definitions stored within an injector system wherein such definitions can then be used during separate operational routines.

### SUMMARY OF THE INVENTION

Those needs identified above and other problems of conventional injector systems are addressed by embodiments of the present invention which allow a service personnel to update syringe information stored within the injector without requiring a replacement of the injector's firmware.

One aspect of the present invention relates to a method within a contrast media injector system for modifying syringe constants. In accordance with this aspect of the invention, an interface is displayed that permits a service technician to configure one or more service-level aspects of the injector system and within this interface, a data collection routine prompts for the syringe constants. In response to the prompts, input is received relating to the syringe constants and a syringe definition is updated based on the received input.

Another aspect of the present invention relates to a contrast media injector system that includes a processor, a non-volatile storage coupled with the processor, and a service application stored within the non-volatile storage configured to execute on the processor. The application itself includes an interface for configuring the injector system, an input routine configured to receive data related to syringe constants, and an updating routine configured to generate a syringe definition based on the data.

A further aspect of the present invention relates to a method for updating an injector system. In accordance with this aspect, a service mode of the injector system is entered and one or more syringe constants are input. Based on the one or more syringe constants, an additional syringe constant is calculated and a syringe definition is stored in the injector system based on the one or more syringe constants and the calculated syringe constant.

### BRIEF DESCRIPTION OF THE DRAWINGS

FIG. 1 illustrates a perspective view of an injector in accordance with principles of the present invention, including a power head, console, and power pack (under a cover), with the syringe, pressure jacket, heater blanket and air detection module removed.

FIG. 2 illustrates a perspective view of the power head of the injector of FIG. 1 with a pressure jacket, syringe and heater blanket mounted thereto, showing the power head display, hand-operated control, and support arm mounting in greater detail.

FIG. 3 illustrates an exemplary graphical user interface that can be presented to a service technician.

FIG. 4 illustrates a flowchart of an exemplary method for updating a syringe definition according to embodiments of the present invention.

### DETAILED DESCRIPTION OF THE INVENTION

Motorized Injection Systems

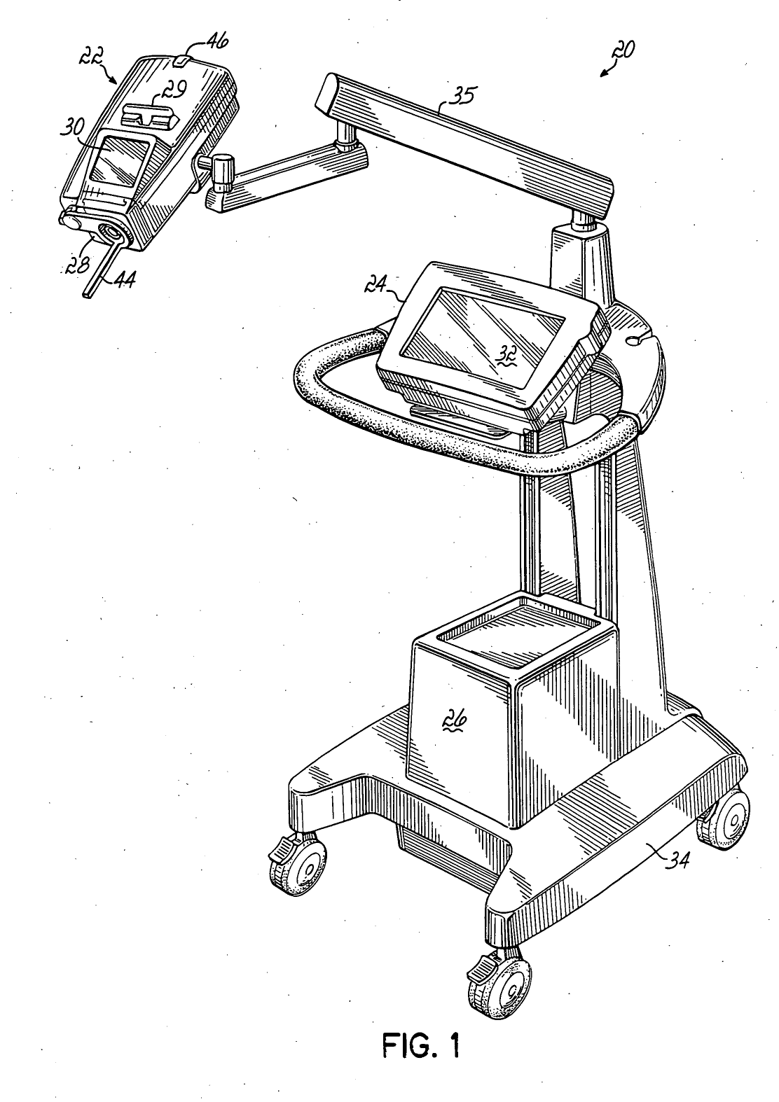

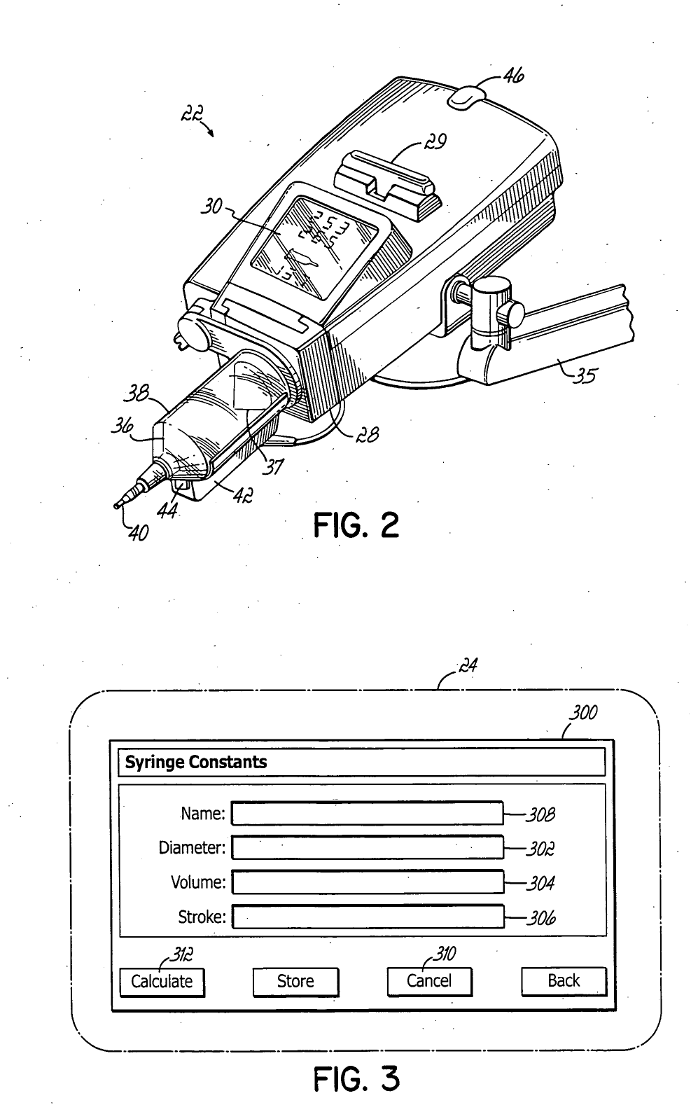

Referring to FIG. 1, an injector 20 in accordance with the present invention includes various functional components, such as a power head 22, a console 24 and power pack 26 (mounted inside of a cover). A syringe 36 (FIG. 2) is mounted to the injector 20 in the face plate 28 of the power head 22, and the various injector controls are used to fill the syringe with, e.g., contrast media for a CT, Angiographic or other procedure, which media is then injected into a subject under investigation under operator or pre-programmed control.

The injector power head 22 includes a hand-operated movement control lever 29 for use in controlling the movement of the internal drive motor, and a display 30 for indicating to the operator the current status and operating parameters of the injector. The console 24 includes a touch screen display 32 which may be used by the operator to remotely control operation of the injector 20, and may also be used to specify and store programs for automatic injection by the injector 20, which can later be automatically executed by the injector upon initiation by the operator.

Power head 22 and console 24 connect through cabling (not shown) to the power pack 26. Power pack 26 includes a power supply for the injector, interface circuitry for communicating between the console 24 and power head 22, and further circuitry permitting connection of the injector 20 to remote units such as remote consoles, remote hand or foot control switches, or other original equipment manufacturer (OEM) remote control connections allowing, for example, the operation of injector 20 to be synchronized with the x-ray exposure of an imaging system

Power head 22, console 24 and power pack 26 are mounted to a carriage 34 which includes a support arm 35 for supporting power head 22 for easy positioning of power head 22 in the vicinity of the examination subject. Other installations are also contemplated however; for example, console 24 and power pack 26 may be placed on a table or mounted on an electronics rack in an examination room while power head 22 is supported by a ceiling, floor or wall mounted support arm.

Referring now to FIG. 2, in operation, a syringe 36 and pressure jacket 38 are mounted to power head 22, so that the motor internal to power head 22 may be energized to move a plunger 37 within the barrel of syringe 36 toward and away from a discharge tip 40 of the syringe, to thereby expel fluid from the syringe 36 or fill the syringe with fluid. Pressure jacket 38 provides support to the outer walls of syringe 36 to protect the walls of syringe 36 from failure at high injection pressures.

Syringe 36 and pressure jacket 38 are made of a clear plastic material through which the operator can view the current location of plunger 37 and any fluid or air in the syringe between plunger 37 and discharge tip 40. Accordingly, as described above, an operator may tilt power head 22 upward, fill syringe 36 from a source of fluid while visually monitoring the filling process, then connect the injector to tubing leading to the patient, expel air from the tubing and syringe while visually monitoring the level of fluid in the syringe, and then once air has been expelled, tilt the injector downward and proceed to inject fluid into a subject.

To facilitate this filling process, and other operations that may be performed during injection of a subject, power head 22 includes the hand-operated movement control, which is in the form of the rotatable lever 29. Specifically, lever 29 is rotatable on an axis of rotation inside of power head 22. When the hand-operated control lever 29 is left in its home position, illustrated in FIG. 2, no plunger motion is generated by power head 22. However, when hand-operated control lever 29 is rotated toward syringe 36, forward plunger motion is generated by power head 22, expelling fluid or air from syringe 36. Alternatively, when hand-operated control lever 29 is rotated away from syringe 36, reverse plunger motion is generated by power head 22, filling syringe 36 with fluid or air.

To ensure that fluid injected into a subject is maintained at approximately body temperature, a heater blanket 42 is installed abutting the exterior wall of pressure jacket 38. Heater blanket 42 includes an electrical heater which generates heat for regulating the temperature of fluid within syringe 36. Heater blanket 42 is mounted to a post 44 extending from face plate 28, holding heater blanket 42 in thermal contact with pressure jacket 38.

At the rear end of power head 22 is an indicator lamp 46 (covered by a light-diffusing cover) which indicates the status of the power head.

Further details of exemplary hardware and software which control operation of an injector system such as that illustrated in FIGS. 1 and 2 can be found in U.S. Pat. No. 5,868,710 which is assigned to the assignee of the present invention and incorporated herein by reference, in its entirety.

Syringe Definitions

As mentioned, the console 24 and touch screen display 32 offer an user interface for an operator of the injector 20. Because the functionality related to maintaining an injector differs from that utilized by an operator, service personnel are typically provided an interface screen on the console different than an operator's interface screen. From this service interface screen, a technician can be offered a menu selection to add, or to modify, the stored definition of a syringe's physical characteristics. The service technician can provide input to the user interface via the input devices (e.g., keyboard, touchscreen, etc.) that are part of the injector 20 or from other diagnostic equipment which can connect to interface ports of the injector 20.

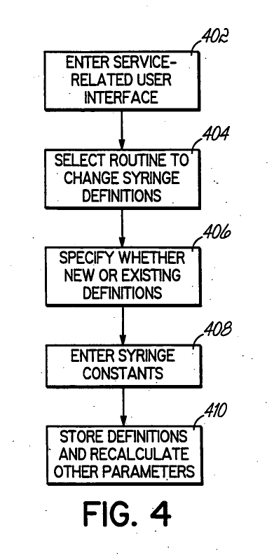

Once the technician elects, through the user interface, to proceed with changing a syringe definition, the technician can be presented an interface screen such as the exemplary screen 300 depicted in FIG. 3. FIG. 4 illustrates an exemplary flowchart for updating syringe definitions.

According to the flowchart of FIG. 4, the service technician uses the console 24 to reach (step 402) the service user interface provided by the injector and selects (step 404), from among a plurality of service-related choices, a routine that permits changing of the stored syringe definitions. This particular service routine permits the technician to specify (step 406) whether the intended change is creating a new syringe definition or changing an existing definition. If changing an existing definition, the technician can be presented with the names of stored syringes to aid with selecting the right definition to update.

Eventually, this particular service routine displays an interface screen 300 that preferably presents the technician with an opportunity to enter (step 408) any of the following syringe parameters or syringe constants: syringe inner diameter, syringe stroke length, and syringe volume. These values are interrelated such that specifying any two of them will permit the third to be calculated automatically. Also, the interface will preferably provide an opportunity for the service technician to label, or otherwise designate, the new syringe information. Doing so will allow an operator to more easily select the correct syringe when operating the injector.

This functionality of step 408 can be provided via a graphical user interface (see FIG. 3) that includes a data entry box 302 for entering a syringe diameter; a data entry box 304 for entering a syringe volume and a data entry box 306 for entering a syringe stroke length. The inclusion of another data entry box 308 will facilitate labeling the syringe definition. Once two values have been input, the service technician can use the button 312 to calculate the third syringe constant according to the relationship that (bore·stroke=volume). Other interface elements 310 permit the service technician to navigate to the other, different service routines available though the service-related user interface.

In practice, a service technician would have the necessary parameters for a new syringe definition provided to them from the syringe manufacturer or distributor. This information could then be used to create a new syringe definition in the injector. Once created, the syringe definition could be stored (step 410) as an additional entry in the non-volatile memory of the injector or can be stored so as to overwrite an outdated entry in the memory.

In the instance in which the service technician is not creating a new syringe definition but, instead, is merely modifying an already existing entry, the interface can populate the data entry screen with the existing values for that syringe definition. The technician can then modify one or more syringe parameters as before. So that the injector system can determine which parameters are newly input and which one is to be calculated, the service technician may clear the one parameter that will be calculated via the interface.

Because the different delivery and filling routines performed by the injector system utilize the syringe parameters from the stored definitions, the injector system 20 will need to ensure that the updated syringe information is used during its operation. As part of storing the updated syringe definition, that routine could recalculate and update all the affected stored parameters that are relied upon by the operational routines of the injector system. Alternatively, the service-level routine can flag any outdated parameters and rely on the operational routines to test parameters to determine their validity and if invalid, recalculate. Thus, upon initializing of an operational routine started by an operator, the injector system 20 can clear any storage of syringe definitions or affected parameters that may now be outdated and read the definitions from the more-permanent storage and recalculate the parameters. Alternatively, the clearing of storage can be dependent on whether the injector system 20 determines, through some type of self-detection routine, whether a service routine has recently been run.

Further details of the wide variety of protocols and routines which a injector system can automatically perform using stored syringe definitions and related parameters can be found in U.S. Pat. No. 5,662,612 which is assigned to the assignee of the present invention and incorporated herein by reference, in its entirety. Accordingly, the stroke length or volume of a syringe used during operational routines can be easily updated by a service technician to accommodate variability of syringes due to manufacturing variances, resin changes, syringe design changes and changing system requirements.

While the present invention has been illustrated by description of various embodiments and while these embodiments have been described in considerable detail, it is not the intention of the applicant to restrict or in any way limit the scope of the claims to such detail. Additional advantages and modifications will readily appear to those skilled in the art. The invention in its broader aspect is, therefore, not limited to the specific details, representative system, apparatus, and method, and illustrative example shown and described. Accordingly, departures may be made from such details without departing from the spirit or scope of the applicant's general inventive concept.

1. Within a contrast media injector system, a method for modifying syringe constants, said method comprising the steps of:
displaying an interface that permits a service technician to configure one or more service-level aspects of the injector system;
providing within the interface a data collection routine that prompts for the syringe constants;
in response to said prompts, receiving input related to the syringe constants; and
updating a syringe definition according to the received input.

2. The method according to claim 1, further comprising the steps of:
storing the syringe definition in a non-volatile memory that is part of the injector system.

3. The method according to claim 1, wherein the syringe constants comprise one or more of a syringe diameter; a syringe stroke length, and a syringe volume.

4. The method according to claim 3, further comprising the steps of:
requesting data related to two of the syringe constants; and
calculating the third syringe constant.

5. The method according to claim 1, further comprising the step of:
modifying one or more operational routines of the injector system affected by the step of updating the syringe definition.

6. The method according to claim 1, further comprising the step of:
modifying one or more parameters stored by the injector system which are affected by the step of updating the syringe definition.

7. The method according to claim 1, further comprising the step of:
associating a label with the syringe information based on the received input.

8. The method according to claim 1, wherein the step of updating includes one of the steps of a) modifying an existing syringe definition or b) creating a new syringe definition.

9. The method according to claim 2, further comprising the step of:
deleting another syringe definition from the non-volatile memory before storing the syringe definition.

10. The method according to claim 1, further comprising the steps of:
exiting the interface;
displaying an operational interface whereby an operational routine is executed.

11. The method according to claim 10, wherein the operational routine relies on the syringe definition.

12. A contrast media injector system comprising:
a processor;
a non-volatile storage coupled with the processor;
an application stored within said non-volatile storage configured to execute on said processor, said application including:
an interface for configuring the injector system;
an input routine configured to receive data related to syringe constants; and
an updating routine configured to generate a syringe definition based on the data.

13. The system according to claim 12, wherein the input routine is further configured to prompt a technician for a portion of the syringe constants; to determine an omitted syringe constant; and to calculate the omitted syringe constant.

14. The system according to claim 12, wherein the syringe definition is stored in the non-volatile storage.

15. The system according to claim 12, further comprising:
an operational application stored in the non-volatile storage, said operational application includes one or more control routines to operate an injector of said injector system, wherein the control routines use the syringe definition to operate the injector.

16. A method for updating an injector system comprising the steps of:
entering a service mode of the injector system;
inputting one or more syringe constants;
based on the one or more syringe constants, calculating an additional syringe constant; and
storing a syringe definition in the injector system based on the one or more syringe constants and the calculated syringe constant.

17. A method for updating an injector system comprising the steps of:
entering a service mode of the injector system;
inputting at least three syringe constants; and
storing a syringe definition in the injector system based on the three syringe constants.

18. The method according to claim 17, wherein the at least three syringe constants comprise a syringe diameter; a syringe stroke length, and a syringe volume.

19. The method according to claim 17, further comprising the step of:
updating a software routine within the injector system which relies on the syringe definition.

20. The method according to claim 17, wherein the one or more syringe constants are selected from the group comprising: syringe diameter, syringe stroke length, and syringe volume.
